(19) United States
(12) Patent Application Publication
(10) Pub. No.: US 2013/0171115 A1
(43) Pub. Date: Jul. 4, 2013

(54) CELL-MEDIATED GENE THERAPY FOR CANCER USING MESENCHYMAL STEM CELLS EXPRESSING A SUICIDE GENE

(71) Applicants: Gang Li, Hong Kong SAR (CN); Yuk Wai Lee, Hong Kong SAR (CN); Po Ying Lau, Hong Kong SAR (CN)

(72) Inventors: Gang Li, Hong Kong SAR (CN); Yuk Wai Lee, Hong Kong SAR (CN); Po Ying Lau, Hong Kong SAR (CN)

(21) Appl. No.: 13/341,239

(22) Filed: Dec. 30, 2011

(51) Int. Cl. A61K 35/12; A61P 35/00; C12N 5/10

(52) U.S. Cl. 424/93.21; 435/366

(57) ABSTRACT

The invention provides cell-mediated gene therapy for cancer using mesenchymal stem cells (MSC) expressing a suicide gene. The MSC are administered to a subject having a tumor, the MSC are allowed to migrate to the tumor site, and then the subject is administered with a prodrug. Expression of the suicide gene by the MSC at the tumor site converts the prodrug to a drug that is lethal to cells of the tumor. The impact of the tumor on the subject's health can thereby be reduced or eliminated. The MSC of this invention may be optimized for use as an off-the-shelf product by using an immortalized MSC line, and selecting MSC with immunological characteristics to forestall a graft-versus-host response.

### FIELD OF THE INVENTION

The invention relates generally to the field of cancer therapy, and to the field of cell isolation and genetic alteration. More specifically, it relates to treatment of cancer using stem cells transfected with a suicide gene such as thymidine kinase.

### BACKGROUND

Cancer is one of the greatest health challenges facing the world today with over 10 million new cases of cancer every year. According to The American Cancer Society, more than 1.4 million new cancer cases were reported for 2007. According to the National Institutes of Health, the annual direct cost of treating cancer patients in the United States is approximately $78 billion. In China, cancer is the number one cause of death and disease. According to IMS Global Insights the Chinese cancer treatment market will be worth more than $40 billion by 2012, driven by an ageing population, better diagnostics, and the introduction of further innovative products that have many years of patent protection ahead of them. The global fight against cancer continues, with more modalities needed to treat cases not amenable to current available anti-cancer agents.

Human adult mesenchymal stem cells (MSCs) were first identified when observing a group of cells that developed into fibroblastic colony forming cells (CFU-F). Since then, therapeutic uses and clinical applications of these cells have been proposed in tissue engineering, gene therapy, transplants and tissue injuries. P M et al., Open Orthop J. 2011;5(Suppl 2):253-60.

Stem cell based cancer therapy has been generally reviewed by M. Cihova et al., Mol Pharmaceutics 2011, 8, 1480-7. MSCs have been found to home to the tumor microenvironment (Mishra P J et al., Cancer Res 2008; 68:4331-9; Hall B et al., Handbook of Experimental Pharmacology 2007; (180): 263-83). They have been used as carriers for in vivo delivery of various clinically relevant anticancer agents, including cytokines Elzaouk L et al., Exp Dermato 2006; 15:865-74), interferon ( )Nakamizo A et al., Cancer Res 2005; 65:3307-18; Studeny M et al., J Nat Cancer Inst 2004; 96:1593-603.), pro-drugs (Miletic H et al., Mol Ther 2007; 15:1373-81; Kucerova L et al., Cancer Res 2007; 67:6304-13.) or replicative adenovirus (Sonabend A M et al., Stem Cells 2008; 26:831-41; Chan J et al., Stem Cells 2005; 23:93-102.). Therapy has also been investigated using genetically modified progenitor cell line in a tumor metastasis model (Aboody K S et al., PLoS ONE 2006;1:e23).

Mesenchymal stem cells and gene therapy was reviewed by Myers T J et al Expert Opin Biol Ther. 2010 December;10(12):1663-79. Therapeutic effect of genetically engineered mesenchymal stem cells has been tested in a rat experimental leptomeningeal glioma model by Gu C et al., Cancer Lett. 2010 May 28;291(2):256-62. Baculovirus-transduced bone marrow MSCs were proposed for systemic cancer therapy by Bak X Y et al., Cancer Gene Ther. 2010 Oct. 17(10):721-9. Use of genetically engineered bone marrow-derived mesenchymal stem cells was proposed for glioma gene therapy by Amano S et al., Int J Oncol. 2009 December;35(6):1265-70. Targeting tumor stroma using engineered mesenchymal stem cells has been proposed to reduce the growth of pancreatic carcinoma by Zischek C et al., Ann Surg. 2009 November;250(5):747-53. Erratum in: Ann Surg. 2010 January;251(1):187. HSV-TK expressing mesenchymal stem cells have been thought to exert bystander effect on human glioblastoma cells, according to Matuskova M et al., Cancer Lett. 2010 Apr. 1;290(1):58-67. Retroviral vector-producing mesenchymal stem cells have been proposed for targeted suicide cancer gene therapy by Uchibori R et al., J Gene Med. 2009 May;11(5):373-81. Potential implications of mesenchymal stem cells in cancer therapy were discussed by Dai L J et al., Cancer Lett. 2011 Jun. 1;305(1):8-20.

Patent publication U.S. 2011/250188 A1 pertains to LCMV-GP-VSV-pseudotyped vectors and tumor-infiltrating virus-producing cells for the therapy of tumors. U.S. 2008/0241115 A1 pertains to use of mesenchymal stem cells genetically modified to express a suicide gene for treating a cancer U.S. 2010/0233200 A1 pertains to a vector encoding therapeutic polypeptide and safety elements to clear transduced cells

Further development of this background research is needed before the technology can be implemented for human therapy on a commercially viable basis.

### SUMMARY OF THE INVENTION

The invention provides a form of cell-mediated gene therapy for cancer using mesenchymal stem cells (MSC) expressing a suicide gene. The MSC of this invention may be optimized for use as an off-the-shelf product by using an immortalized MSC line, and selecting MSC with immunological characteristics to forestall a graft-versus-host response.

A method for treating a tumor in a subject according to this invention typically has the following steps: a) administering to the subject a population of mesenchymal stem cells (MSC) that recombinantly express a suicide gene; b) waiting a sufficient time for at least some of the mesenchymal stem cells to migrate from where they were administered to a location at or around the tumor; and then c) administering to the subject an effective amount of a prodrug, whereby expression of the suicide gene by the mesenchymal stem cells that are located at or around the tumor causes the prodrug to be converted to a drug that is lethal to cells of the tumor.

For use as an off-the-shelf product, the cells may be characterized as “pleiotropic”. This term refers to cells that are sufficiently non-immunogenic that they may be given to subjects in a population having diverse genotype without regard to tissue matching. In a majority of the population, an effective proportion of the mesenchymal stem cells will migrate to the tumor without being removed or rendered inactive by the immune system of the subject. Pleiotropic cells can be included in an off-the-shelf pharmaceutical product without knowing in advance what subjects are to be treated, or their respective tissue type.

Often, the MSC are from a cell line that has been immortalized, for example, by transfecting with SV40 large T antigen using a vector such as a lentivirus. Preferably, the MSC are fetal mesenchymal stem cells or cells from another source that constitute a pleomorphic immunological profile. A model suicide gene is a thymidine kinase (for example, from HSV), for which the prodrug is ganciclovir. Other suicide genes and prodrugs are listed later in this disclosure.

Another embodiment of the invention are products for use as pharmaceuticals, or for use in the preparation of pharmaceuticals. Such products include a line of mesenchymal stem cells that recombinantly express suicide gene. Preferably, the MSC are fetal mesenchymal stem cells or cells from another source that constitute a pleomorphic immunological profile. Again, the MSC are often from a cell line that has been immortalized, for example, by transfecting with SV40 large T antigen using a vector such as a lentivirus. The cells can be transfected with the suicide gene concurrently with the immortalization, or as a separate step.

Such cell lines can be used in the manufacture of a pharmaceutical composition for treating cancer. The composition will include a population of MSC of this invention, and a physiological compatible buffer or other excipient. The invention also provides a kit for treating a tumor in a subject. The kit may contain a pharmaceutical composition of this invention, and the corresponding prodrug, where expression of the suicide gene in the MSC of the composition causes the prodrug to be converted to a drug that is lethal to tumor cells. Such pharmaceutical compositions and kits will typically be packaged with prescribing instructions for use in accordance with a method of this invention.

Other embodiments of the invention will be apparent from the description that follows.

### DESCRIPTION OF THE DRAWINGS

FIG. 1 shows stem cell characteristics of human fetal BMSC. 1(A) shows single colony forming ability. 1(B) shows multiline age differentiation potential of human fetal bone marrow MSC compared with human adult MSC.

FIG. 2 shows surface markers of human fetal bone marrow derived MSC determined by flow cytometry.

FIG. 3 provides gene maps of an SV40 plasmid which encodes large and small T antigen in transduced BMSC (Top), and a TK and GFP co-expressing plasmid (Bottom).

FIG. 4 shows a colony of MSC (Top), expression of SV40 determined by Western blot (Middle) and expression of SV40 in four batches of transduced MSC (Bottom).

FIG. 5 shows a GFP-positive colony of MSC (Top), gene expression by picked colonies (Middle), and morphology of SV40-TK-GFP expressing MSC.

FIG. 6(A) shows exemplary surface markers of gene transduced MSC of the invention.

FIG. 7(A) shows a workflow for the sorting of MSC after transduction.

FIG. 8 shows osteogenic and adipogenic differentiation ability of MSC after transduction.

FIG. 9 shows cell proliferation of MSC after transduction.

FIG. 10 shows results of a study of the transwell migration ability of hfBMSC in the presence or absence of human prostate cancer cells.

FIG. 11 shows in vitro cytotoxicity of GCV on human prostate cancer cell line (DU145) in a co-culture with SV40-TK-GFP human fetal MSC.

FIG. 12 shows results of an MLR assay on the transduced MSC.

FIG. 13 shows in vivo anti-tumor effect of the MSC of this invention.

### DETAILED DESCRIPTION

This invention provides pharmaceutical compositions and therapeutic methods for cancer treatment. Mesenchymal stem cells (MSC) are engineered to express a suicide gene that converts a prodrug to a drug that is lethal to tumor cells. The MSC are administered to a subject having a tumor, whereupon the MSC migrate to the tumor site. The subject is then administered with a prodrug, which is converted to the lethal form at or near the tumor by the MSC expressing the suicide gene. The impact of the tumor on the subject's health can thereby be reduced or eliminated.

This invention provides a number of improvements over previous mesenchymal stem cell (MSC) based treatment modalities. The improvements can be used separately or in combination to produce an off-the-shelf product with well-characterized stem cell properties, tumor tropism, low immunogenicity, capable of delivering suicide gene to tumor microenvironment, and having proliferative capacity suitable for bulk production

Previous work with MSC has typically relied on freshly isolated or cultured cells, which are then given to subjects that are syngeneic or isogeneic with the cell preparation. As a practical matter, MSC of this invention may be immortalized as a standardized source of cells having known phenotype that can be scaled up for commercial use. It has been discovered that by immortalizing and optionally selecting the cells in accordance with this invention, it is possible to establish mixed or clonal cell lines with ongoing proliferative capacity, yet retaining the ability to home to tumor cells and thereby deliver the effective agent to the target.

It has also been discovered that obtaining relatively early MSC, cell populations can be obtained that are relatively non-immunogenic in the potential subject population for the pharmaceuticals. The cells can thus be given to individuals having a wide range of differing tissue types. This means that they can be prepared in advance of the therapy, without knowing the intended recipient. Pharmaceutical compositions having this property may be referred to as an off-the-shelf product.

### Possible Sources and Characterization of Stem Cells

The founding population of cells for an MSC preparation of this invention can be taken from an ontogenetically primitive source. The experimental investigation done during the making of this invention showed that this provides several advantages. First, the cells may adapt better to tissue culture, have greater proliferative capacity, and may be more amenable to genetic alteration and immortalization.

Second, ontogenetically primitive MSCs generally have the desired property of being relatively non-immunogenic across a wide range of tissue types. Unless MSC are autogeneic to the intended recipient, administration of MSC to humans will almost inevitably involve differences in tissue type between the cell line and the recipient. Typically, tissue allografts will result in a Type IV graft-versus-host reaction in an immunologically naïve subject, or a hyperacute rejection in subjects having preformed antibody. An immunological response may eliminate the cells before they react the target tumor cells in the recipient, or block the effectiveness of subsequent doses of the same cells.

However, early MSC used as a founding population for a cell line or pharmaceutical composition of this invention may be immunoprivileged in the recipient, or evoke a muted response. This is demonstrated in the Examples below, in which human MSC can be used in a xenograph to an animal model, and still be effective in delivering the suicide gene to the site of the tumor.

Exemplary starting MSC can be obtained from second or third trimester fetal tissue: particularly from bone marrow (Example 1). In principle, fetal MSC may also be obtained from the embryonic mesenchyme, the mesoderm, cord blood, fetal tissue homogenates or other fetal tissues. Human multipotent mesenchymal stem cells can also be isolated from amniotic fluid (Tsai M S et al., Hum Reprod. 2004 June;19(6):1450-6).

The MSC may be characterized according to both functional properties and phenotypic markers. For example, they may be CD34 −ve, CD44 +ve, CD73 +ve, and CD90 +ve (Example 3). U.S. 2006/0166214 A1 provides a marker system for detecting mesenchymal stem cells and a method of distinguishing mesenchymal stem cells. See also the review of cell surface markers for mesenchymal stem cells by P M et al., Open Orthop J. 2011;5(Suppl 2):253-60. MSC populations may also be characterized by functional features, such as the ability to differentiate into osteogenic, adipogenic, or chondrogenic tissue; and/or the ability to home to tumor cells.

None of these features are absolutely required, as long as the cells have the properties of tumor homing and suicide gene expression. However, phenotypic markers may be used in cell preparation to sort desirable MSC from other cell types. They may also be used as quality control, for example, at the time of initial isolation, after a period in tissue culture, after immortalization, after transduction to express a suicide gene, and after formulation as a pharmaceutical product.

### Immortalization

Once the source MSC have been adapted to tissue culture, they may be treated so as to increase proliferative capacity and/or replication rate.

Several methods are available for immortalizing mammalian cells in culture. Viral genes, including Simian virus 40 (SV40) T antigen, Epstein Barr virus (EBV), Adenovirus E1a and E1b, and human Papilomavirus (HPV) E6 and E7 can induce immortalization in different cell types. For the most part, viral genes achieve immortalization by inactivating the tumor suppressor genes (p53, Rb and others) that can induce a replicative senescent state in cells.

Another approach to cell immortalization is through the expression of telomerase Reverse Transcriptase protein (TERT). This protein is inactive in most somatic cells, but when hTERT is exogenously expressed, the cells are able to maintain telomere lengths sufficient to avoid senescence. Analysis of several telomerase-immortalized cell lines has verified that the cells maintain a stable genotype and retain critical phenotypic markers: see U.S. Pat. No. 7,195,911.

SV40 T antigen has been shown to be a reliable agent for the transformation of many different cell types in culture. Rather than infecting with SV40 itself, the cells can be immortalized by transfecting critical SV40 genes in a delivery vector such as lentivirus. Optionally, the same vector can be used concurrently to deliver the suicide gene (infra), or a marker gene such as green fluorescence protein (GFP). Suitable vectors are available commercially. For example, Capital Biosciences supplies a recombinant Lentiviral Vector containing SV40 large and small T antigen, and expressing mutant pLenti-SV40 gene for use in gene-specific cell immortalization.

Once the MSC have been treated in such a fashion, they may be tested for proliferative capacity, for example, using a standard BrdU assay (Example 3). If the replicative properties of the cell are not optimal, the cell population can either be subject to further rounds of immortalization. It is also possible to enrich for cells with increased proliferative capacity by transfecting cells simultaneously with a gene such as SV40 T antigen, and with a cell marker. For example, the marker can be encoded in the same vector, separated from the SV40 antigen by an IRES. If the marker is chemiluminescent or bioluminescent like green fluorescent protein (GFP), cells can be enriched by fluorescence-activated cell sorting. Alternatively, if the marker is a unique cell-surface antigen, transfected cells can be enriched by using a fluorescent antibody specific for the marker. The user may subject the cells to rounds of immortalization and enrichment in any combination as desired.

Transduction with the Suicide Gene

The MSC of this invention are genetically altered to express a suicide gene. Any suitable vector may be used where the suicide gene is expressed under control of a promoter that is active in the target tissue or tumor cell. The promoter may be chosen to be active in all cells, such as a CMV promoter. Alternatively, the promoter may be chosen so that the suicide gene is tissue-specific. This means it is expressed only in cells that are characteristic of the target. For example, pharmaceutical agents targeted to prostate cancer may comprise a suicide gene under control of the PSA promoter. The vector may optionally include an immortalization component, such as an SV40 T antigen, so that the immortalization and transduction with the suicide gene can be performed in one step. The vector may optionally include a luminescent or cell-surface marker to assist in subsequent enrichment of transduced cells expressing the suicide gene.

Once a cell population has been established that expresses a suicide gene at a sufficient level, its efficacy can be tested in vitro by combining with a population of cells from a cancer cell line (preferably of the same type as the intended cancer target in the population of subjects to be treated). After the MSC have had a chance to express the suicide gene, the prodrug is added to the culture, and the resultant toxicity to the cancer cells is determined. The efficacy can be tested in vivo using a suitable animal model (Example 7), in accordance with the treatment methods of this invention.

The term “suicide gene” in the context of this invention means a gene that expresses an enzyme capable of converting a particular prodrug to a drug that is toxic to the target tumor cell. The drug may or may not be toxic to the MSC that delivers it to the tumor site.

Although thymidine kinase (TK) from HSV has been used as a prototype suicide gene of this invention, there are other alternatives. Depending on the target cell type, the desired effect, and suitability of the prodrug, the gene-drug combinations shown in Table 1 can be tested and developed in accordance with this invention. (Table adapted from W. A. Denny, J. Biomedicine Biotechnol. 1:48-70, 2003).

TABLE 1
Suicide Genes and Corresponding Prodrugs

| SUICIDE GENE | PRODRUG |
|---|---|
| HSV thymidine kinase (TK) | Ganciclovir (GCV) |
| | Canciclovir elaidic acid seter |
| | Penciclovir (PCV) |
| | Acyclovir (ACV) |
| | Valacyclovir (VCV) |
| | (E)-5-(2-bromovinyl)-2′- |
| | deoxyuridine (BVDU) |
| | Zidovuline (AZT) |
| | 2′-exo-methanocarbathymidine |
| | (MCT) |
| Cytosine Deaminase (CD) | 5-fluorocytosine (5-FC) |
| Purine nucleoside phosphorylase | 6-metnylpurine deoxyriboside (MEP) |
| (PNP) | fludarabine (FAMP) |
| Cytochrome p450 enzymes (CYP) | Cyclophosphamide (CPA) |
| | Ifosfamide (IFO) |
| | 4-ipomeanol (4-IM) |
| Carboxypeptidases (CP) | 4-[(2-chloroethyl)(2- |
| | mesyloxyethyl)amino]benzoyl- |
| | L-glutamic acid (CMDA) |
| | Hydroxy- and amino-aniline |
| | mustards |
| | Anthracycline glutamates |
| | Methotrexate α-peptides (MTX-Phe)) |
| Carboxylesterase (CE) | Irinotecan (IRT) |
| | Anthracycline acetals |
| Nitroreductase (NTR) | dinitroaziridinylbenzamide CB1954 |
| | dinitrobenzamide mustard SN23862 |
| | 4-Nitrobenzyl carbamates |
| | Quinones |
| Horse radish peroxidase (HRP) | Indole-3-acetic acid (IAA) |
| | 5-Fluoroindole-3-acetic acid (FIAA) |
| Guanine Ribosyltransferase | 6-Thioxanthine (6-TX) |
| (XGRTP) | |
| Glycosidase enzymes | HM1826 |
| | Anthracycline acetals |
| Methionine-α,γ-lyase (MET) | Selenomethionine (SeMET) |
| Thymidine phosphorylase (TP) | 5′-Deoxy-5-fluorouridine (5′-DFU) |

### Use in Treatment

An MSC population that expresses a suicide gene may be used for the treatment of cancer or the eradication of tumor cells in a subject in need thereof.

The MSC are first taken from their source and prepared for administration (for example, by washing as necessary, and/or exchange into administration buffer). They are then administered to the subject in a suitable medium, such as an isotonic saline solution or physiologically compatible buffer, optionally containing other excipients such as thickeners or agents that may assist in cryopreservation. The mode of administration may be systemic (intravenously or subcutaneously) or locally by injection or infusion through an implanted catheter.

The prodrug may be given at any suitable time, typically once at least a proportion of the MSC (perhaps 3%, 5%, 10%, 20% or more) have migrated from the site of administration to a location that is at or near the site of the tumor cells.

The dosage of both the MSC and the corresponding prodrug are adjusted for optimal effect on the tumor with minimal side effects. By extrapolation from effective dose in animal models (0.2 million cells per mouse) the effective dose of MSC in human subjects may be of the order of 1×10⁷ cells per kg, or between 0.2×10⁷ and 5×10⁷ per kg for systemic administration, scaled down appropriately if given locally. The effective dose of a prodrug like GCV may be of the order of 30 mg/kg, or 5 to 150 mg/kg. Ultimate choice of the mode of administration and dosage will be made by the attending physician.

In principle, any form of cancer may be treated according to this invention, subject to appropriate testing beforehand. Since MSC home to tumor cells, the methods of this invention may be adaptable for treatment of unresectable tumors, or metastatic tumors. Treatment according to this invention may have any one or more of several desirable endpoints. These include decrease or cessation of tumor growth, decrease in symptoms experienced by the patient, decrease in serum markers for the target cancer, eradication of the tumor, and increase in life expectancy.

This invention also provides systems and kits for treating multiple patients, and/or treating one patient on several occasions. These systems and kits comprise multiple cell lines having a different tissue type, genetically altered to express the same or different suicide genes. Typically, MSC are taken from a source having blood group O so as to be useable with subjects having any ABO blood group. However, different MSC preparations may be generated from founder cells from any of the blood groups for administration to patients where the blood group is the same.

Where the MSC have a low monogenetic profile, tissue matching is typically not necessary. However, where desirable (for example, where the subject has already been sensitized to certain allotypes), the clinician may wish to do a cytotoxicity test (MSCs in patient serum) and/or tissue matching for histocompatibility Class I and/or Class II markers. A particular preparation of MSC is then chosen from the system so as to minimize allogenicity and/or hyperacute rejection. When multiple doses are required, the clinician may wish to select MSCs having a different tissue type each time, in case the previous dose sensitized the subject to the tissue type(s) used for previous doses.

### Definitions

The term “mesenchymal stem cells” (MSC) is used in this disclosure according to its standard meaning in the art. The term generally refers to multipotent stem cells that can differentiate into particular cell types, exemplified by osteoblasts (bone cells), chondrocytes (cartilage cells) and adipocytes (fat cells), and bear characteristic markers of mesenchymal stem cells, such as CD44, CD73, and CD90.

“Pleiotropic” cells refers to cells that are sufficiently non-immunogenic that they may be given to subjects in a population having diverse genotype without regard to tissue matching. In a majority of the population, an effective proportion of pleiotropic mesenchymal stem cells will migrate to a tumor without being removed or rendered inactive by the immune system of the subject.

A cell may be described as “immortalized” if it has been adapted by the hand of man (for example, by transfection with an SV40 T antigen) to increase its proliferative capacity either to at least 50 doublings, or to at least three times the Hayflick limit of the cell before the adaptation.

The term “suicide gene” in the context of this invention means a gene that expresses an enzyme capable of converting a particular prodrug to a drug that is toxic to the target tumor cell. The drug may or may not be toxic to the MSC that delivers it to the tumor site.

The term “prodrug” refers to a chemical compound that is substantially inert in its initial structural form, but is convertible by a specific enzyme to a modified structure with physiologically significant reactivity—such as a compound that is toxic to tumor cells.

A method that refers to “treating” a subject with a particular composition is a method wherein the stated composition is administered to the subject with the objective of reducing the pathology, symptomatology, or undesirability that is associated with a certain disease or condition—such as the presence in the subject of a tumor or cancerous tissue. In any particular instance, the subject may or may not benefit from the treatment.

An “effective dose” or “effective amount” of a pharmaceutical composition is an amount of the composition which (when given in the manner stated) is effective in reducing the pathology, symptomatology, or undesirability of a stated disease or condition in a majority of subjects in the same situation and phenotype, and having the same condition as the subject actually being treated.

### General Techniques

General methods in molecular genetics and genetic engineering are described in the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Tissue culture supplies and reagents are available from commercial vendors such as Gibco/BRL, Nalgene-Nunc International, Sigma Chemical Co., and ICN Biomedicals.

Cells prepared according to this invention that are useful for human or veterinary therapy are optimally supplied in a pharmaceutical composition, comprising a suitable excipient prepared under sufficiently sterile conditions for human or veterinary administration. For general principles in medicinal formulation, the reader may refer to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The compositions may be packaged with written prescribing information for use of the cells in cancer therapy, in accordance with the methods of this invention. Cells may be kept frozen or in another stable storage means that preserves the desired features of the cells until the time of use.

The examples that follow are provided by way of further illustration, and are not meant to imply any limitation in the practice of the claimed invention or its equivalents.

### EXAMPLES

### Summary

In the illustrations described in detail below, human mesenchymal stem cells (MSCs) were obtained from fetal bone marrow, with ethical approval for the collection of clinical waste from Surgical Termination of Pregnancy. The cells were isolated, identified and cultured in vitro. The cultured cells showed adherence to plastic surface, expression of MSCs-specific surface markers, and multi-lineage differentiation potential.

The MSC were then transfected with the large T and small T antigens of Simian virus 40 (SV40). Cells bearing this gene had a greater proliferation rate as compared with the wild type. The transfer of thymidine kinase (TK) was done simultaneously. The plasmid also contained enhanced green fluorescent protein (GFP) as reporter gene. Thus, it was possible to determine the TK gene transfer efficiency by microscopic observation. Additional rounds of lentiviral infection was carried out to establish a cell line with desirable characteristics.

The transduced cell lines were found to retain important properties, including colony forming ability, MSC specific surface phenotypes, and multiline age differentiation. The proliferation rate of the SV40-TK-GFP human fetal BMSC (bone marrow derived MSC) was maintained. An in vitro luciferase enzyme activity study demonstrated that co-culture of SV40-TK-GFP human fetal BMSC and addition of GSV could significantly induce cytotoxicity in two human prostate cancer cell lines.

A subsequent in vivo study showed that systemic injection of the BMSC (0.2 million cells per mouse) and subsequent intraperitoneal injection of GCV (30 mg/kg) could reduced the tumor mass and luciferase activity of tumor cells in an animal model, compared with negative control.

### Example 1

### Characteristics of Fetal Mesenchymal Stem Cells

The use of human fetal tissues is approved by Joint CUHK-NTEC Clinical Research Ethics Committee (ethical approval code: CRE-2011.383). Fetus upper and lower limbs are transported in PBS with 10% PS. External muscles/membranes/tissues/vessels are removed from the bone. The bone is washed with DPBS twice throughout. Completed Knockout DMEM (KO-DMEM) is added and the bone is cut into small pieces. Bone pieces are further diced and all (include the medium) are transferred to 50 ml centrifuge tube. The tube is inverted several times to elute remaining cells. Cell suspension is collected through a 70 um cell strainer. Cells are pelleted by spinning at 400 g, 5 min, 20° C. and are cultured in KO-DMEM supplemented with 10% FBS, 10% penicillin & streptomycin, and 1% glutamax. 30 ml culture medium is used for cells seeded into T175 culture flask, 15 ml for T75 culture flask, and 5 ml for T25 flask.

For crypopreservation of human fetal bone marrow-derived MSC, the cells collected by trypsinization are washed twice by DPBS. Cells are pelleted by spinning (400 g, 5 min, 4° C.), resuspended in chilled 65% plain KO-DMEM, 30% FBS and 5% DMSO, and transferred into 1.8 ml cryovials with proper labeling, including name of experimentalist, code for the batch of cells, name of cells, passage number, cell number, and date of experiment. The cells are frozen in a controlled rate in which temperature drops from 4° C. to −80° C. at 1° C./min. The cells are then transferred to liquid nitrogen tank for long term storage.

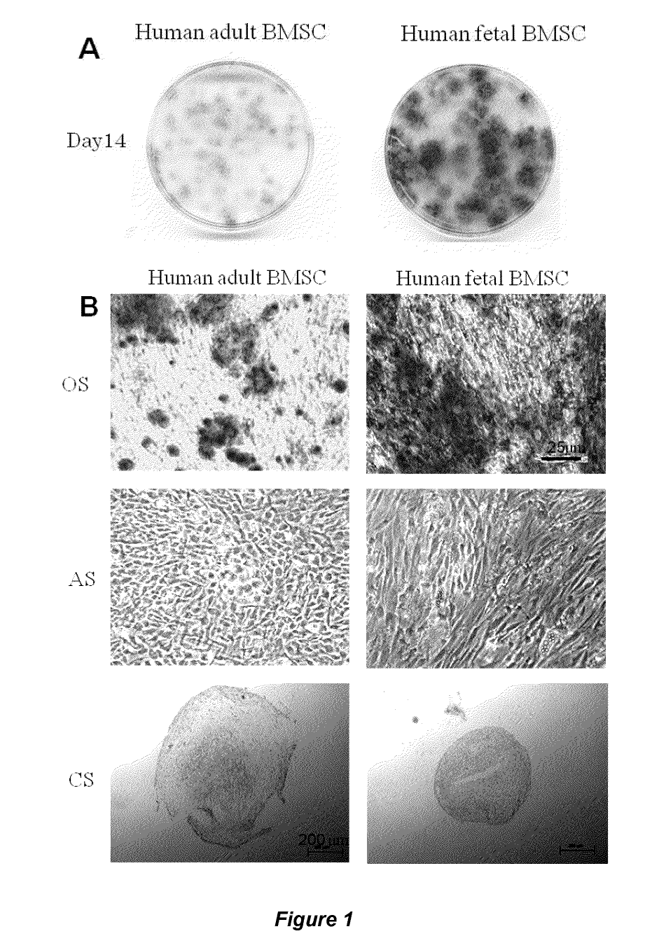

FIG. 1 show stem cell characteristics of human fetal BMSC. FIG. 1(A) Single colony forming ability at day 14 after low density plating (50 cells/100 mm dish) and stained by crystal violet. FIG. 1(B) Multilineage differentiation potential of human fetal BMSC as compared with human adult BMSC. For the osteogenic differentiation (OS), the cells were seeded until confluent and incubated in the presence of induction medium for 14 days and stained by alizarin red. For the adipogenic differentiation (AS), the cells were seeded until confluent and incubated in the presence of induction medium for 14 days and stained by oil red O. For the chondrogenic differentiation (CS), the pellet culture (5×10⁵) were incubated in the presence of induction medium for 21 days and stained by Safranin O and counterstained by fast green.

Flow cytometry for surface phenotypes was conducted as follows: 1×10⁶/mL cell suspension is prepared and washed twice with PBS. 100 ul of cell suspension is incubated in fluorochrome-conjugated primary antibodies against CD34, CD44, CD45, CD73, CD90, CD105, and isotypes correspond according to manufacturer's instruction. The stained cells are subjected to flow cytometric analysis.

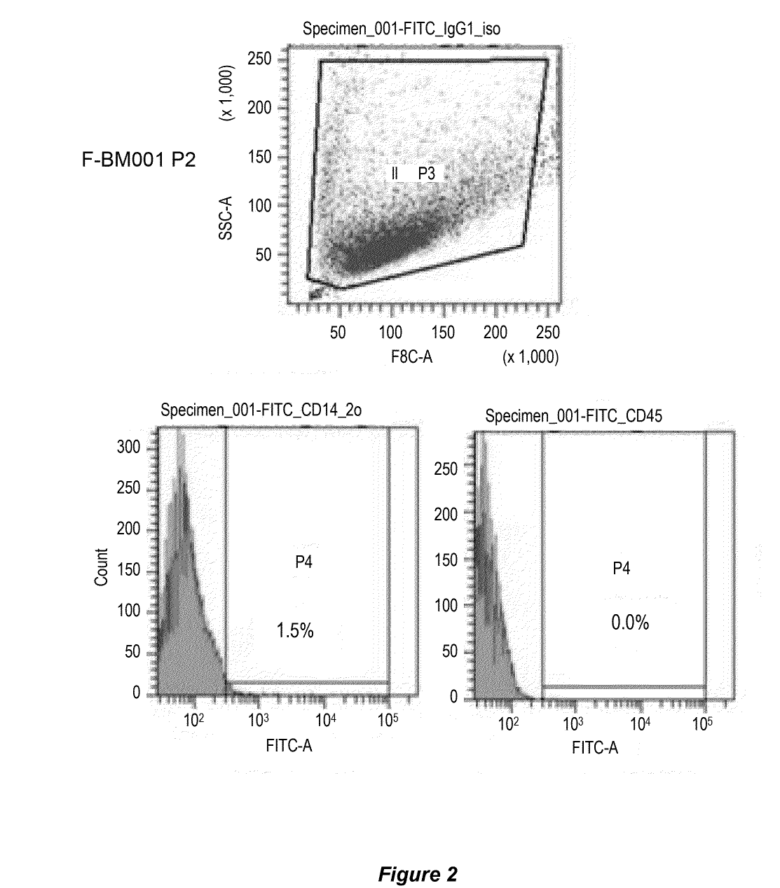

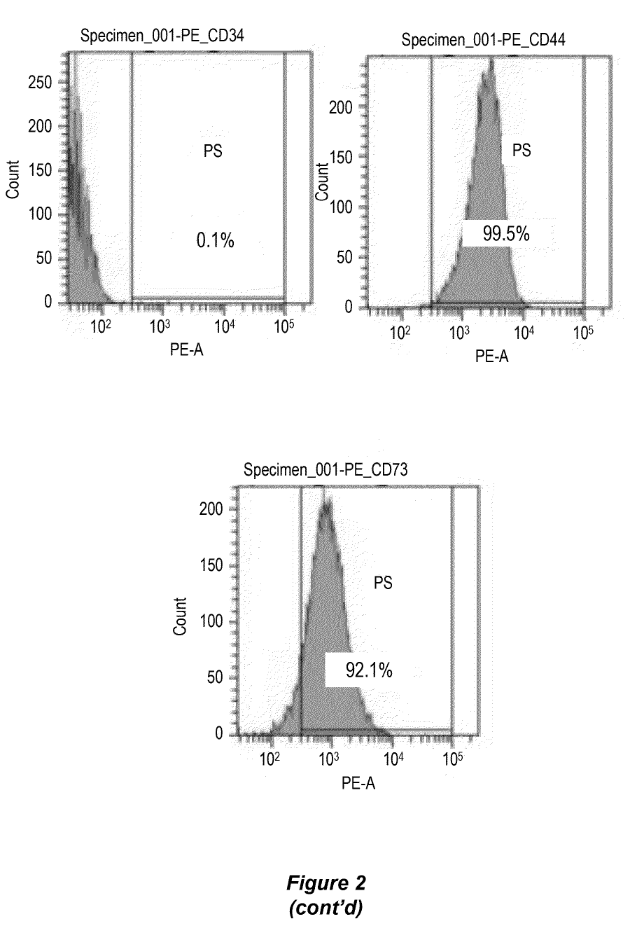

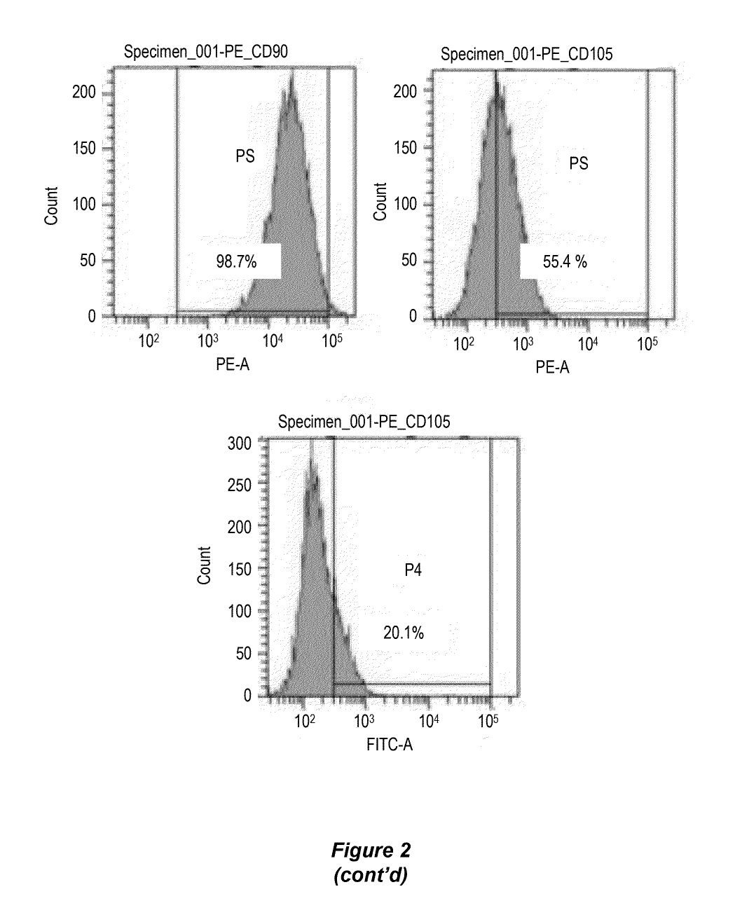

FIG. 2 shows surface markers of hfBMSC determined by flow cytometry.

### Example 2

### Immortalization of the Cells and Transduction with the Suicide Gene

Lentivirus production and gene transduction was done as follows. 1×10⁶ human embryonic kidney cells (298FT cells) are plated into 100 mm dish. At 60% cell confluence, transfer vector plasmid (either pLenti CMV/TO SV40 small+Large T or pLOX-GFP-IRES-TK) DNA (8 μg), helper plasmid plp-1 DNA (5.28 μg), plp-1 DNA (4 μg), and envelope plasmid plp-VSVG DNA (2.8 μg) are added. Transfection is done by calcium phosphate in the presence of 1× HBS. Medium is changed 8 h after the transfection. Virus-containing medium is collected 72 h later, filtered through 0.45 μm filter and stored at −80° C. until use. Gene transduction is carried out by adding the filtered virus-containing medium into MSC (2000 cells/well in 12-well plate), and facilitated by centrifugation at 1000 g for 1 h at 35° C. The medium is changed 18 h later.

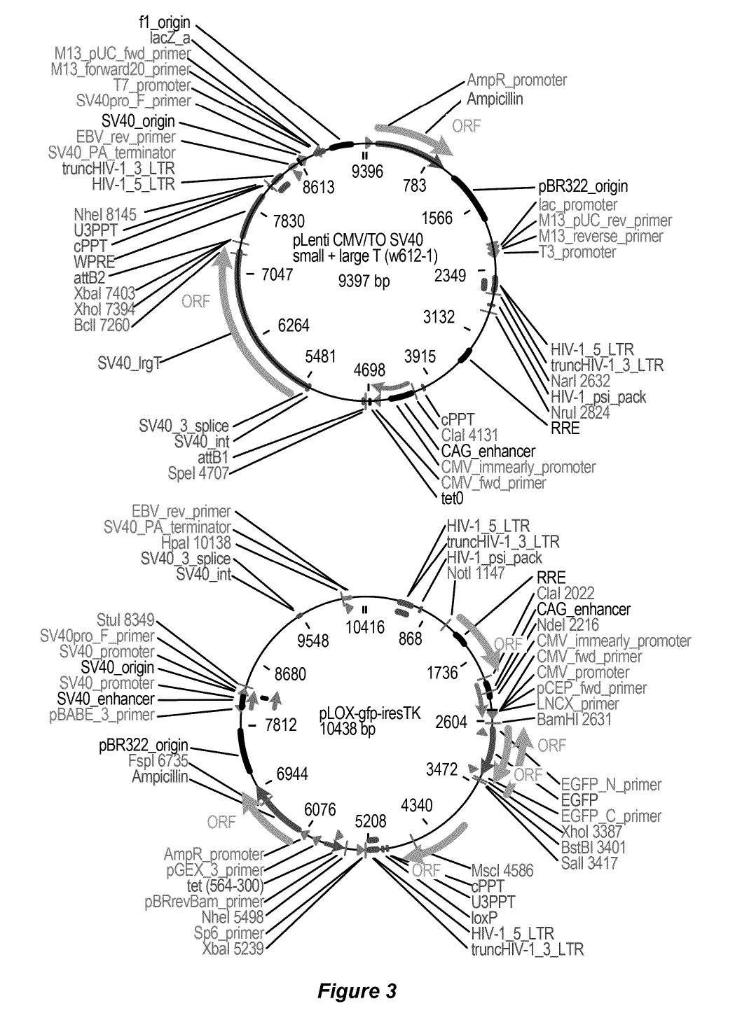

FIG. 3 (Top) shows a schematic structure of SV40 plasmid which encodes large and small T antigen in transduced BMSC, which induced cell replication. FIG. 3 (Bottom) shows a schematic structure of HSV1-TK and GFP co-expressing plasmid which encodes HSV1-TK and GFP in transduced BMSC.

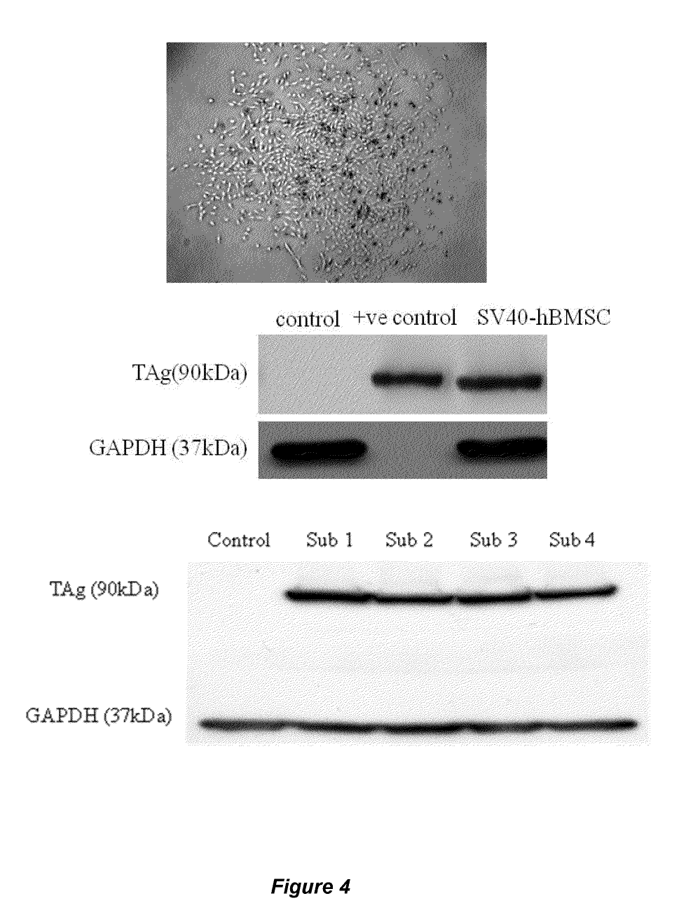

FIG. 4 (Top) shows a single colony of human fetal BMSC after transduction of SV40 lentivirus. FIG. 4 (Middle) shows expression of SV40 in SV40-transduced hfBMSC as determined by Western blot. FIG. 4 (Bottom) shows expression of SV40 in four batches of SV40-transduced hfBMSC.

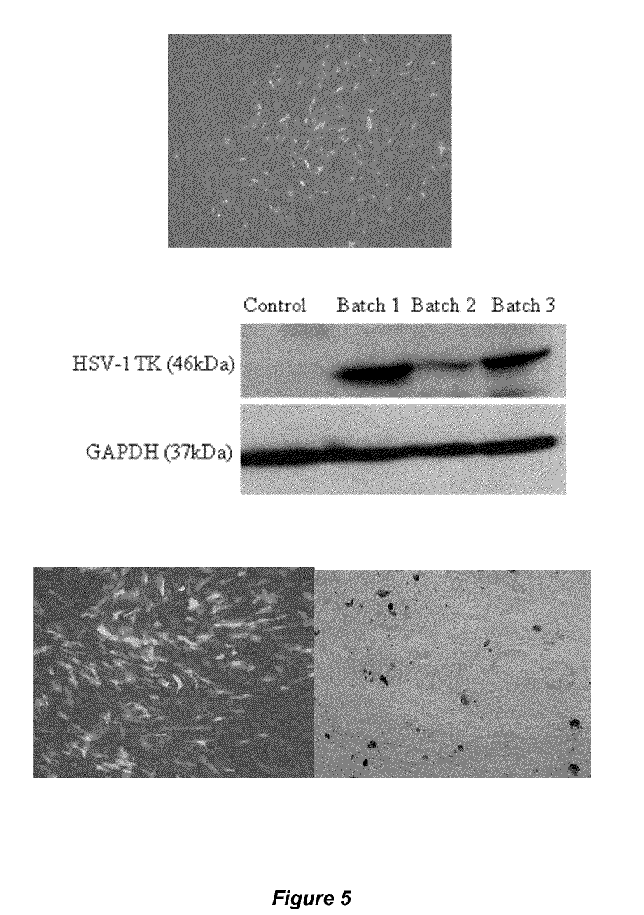

FIG. 5 (Top) shows a GFP-positive colony of SV-human fetal BMSC after transduction of HSV1-TK and GFP co-expressing lentivirus. FIG. 5 (Middle): Several colonies were picked, and expanded to sufficient number for Western blot. FIG. 5 (BOTTOM): Representative image of batch of SV40-TK-GFP-human fetal BMSC with higher protein level of HSV1-TK was selected for further expansion.

### Example 3

### Characterization and Sorting of Transfected MSC

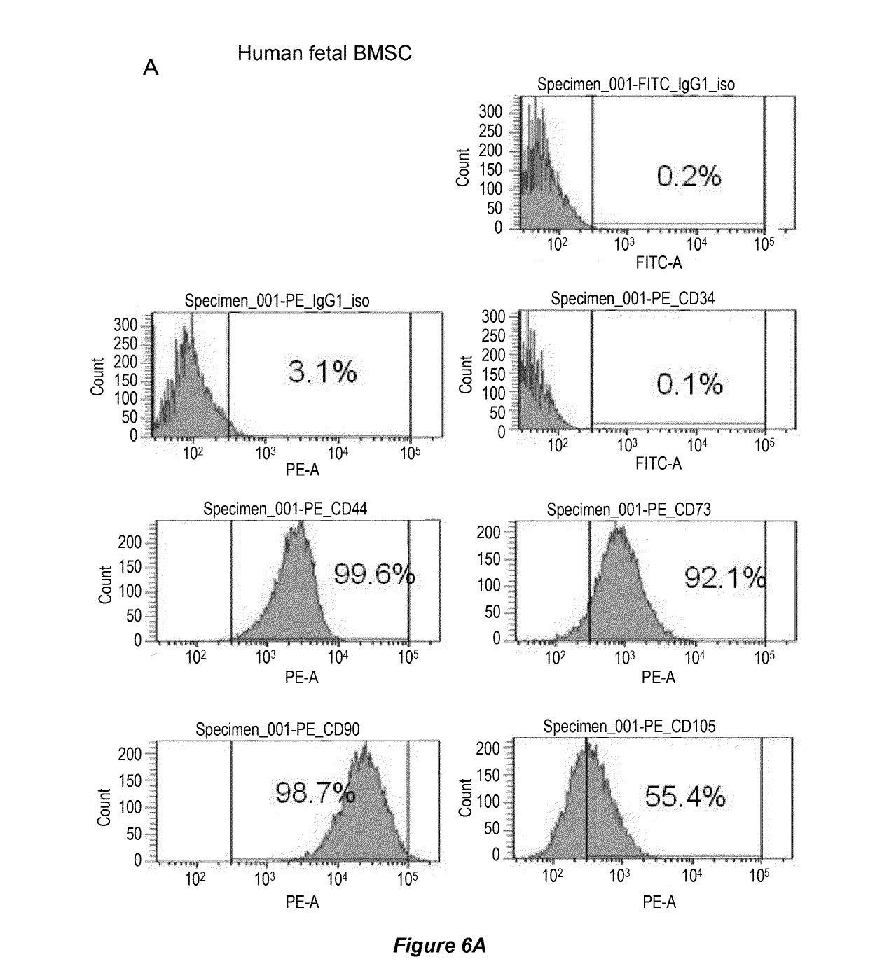

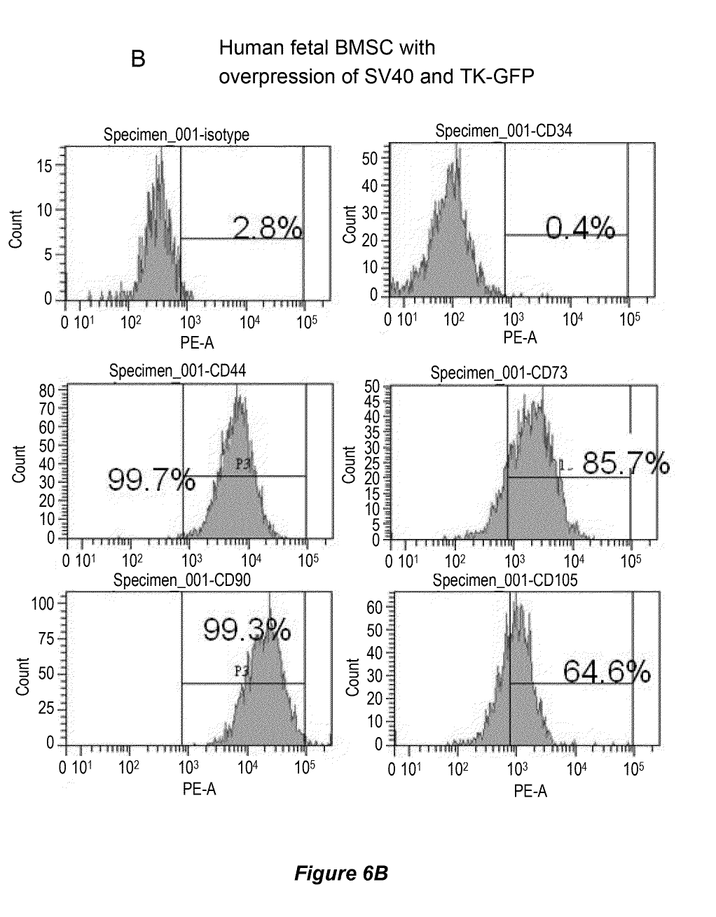

FIG. 6(A) shows surface markers of hfBMSC detected by flow immunocytometry. FIG. 6(B) shows surface markers of SV40-TK-GFP-overexpressing hfBMSC. In hfBMSC, anti-CD34 conjugated with FITC was used, thus, a isotype control for this FITC-conjugated antibody. While for the GFP-positive transduced hfBMSC, anti-CD34 conjugated with PE was used for 2-color detection. The cells were CD34 −ve, CD44 +ve, CD73 +ve, CD90 +ve, and CD105 partly positive.

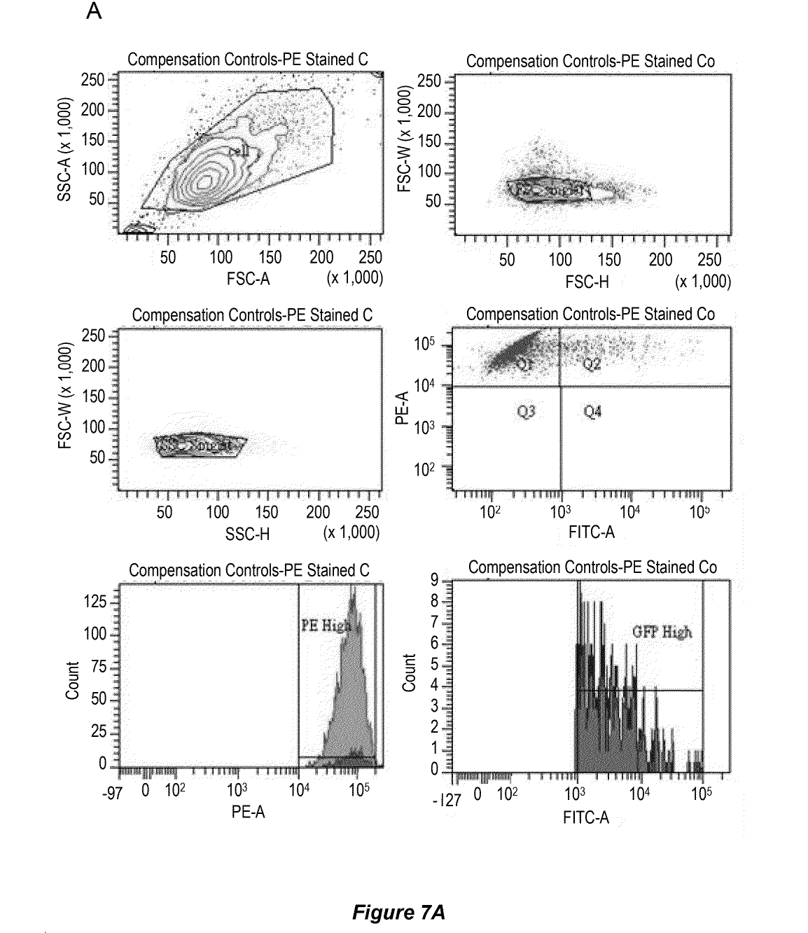

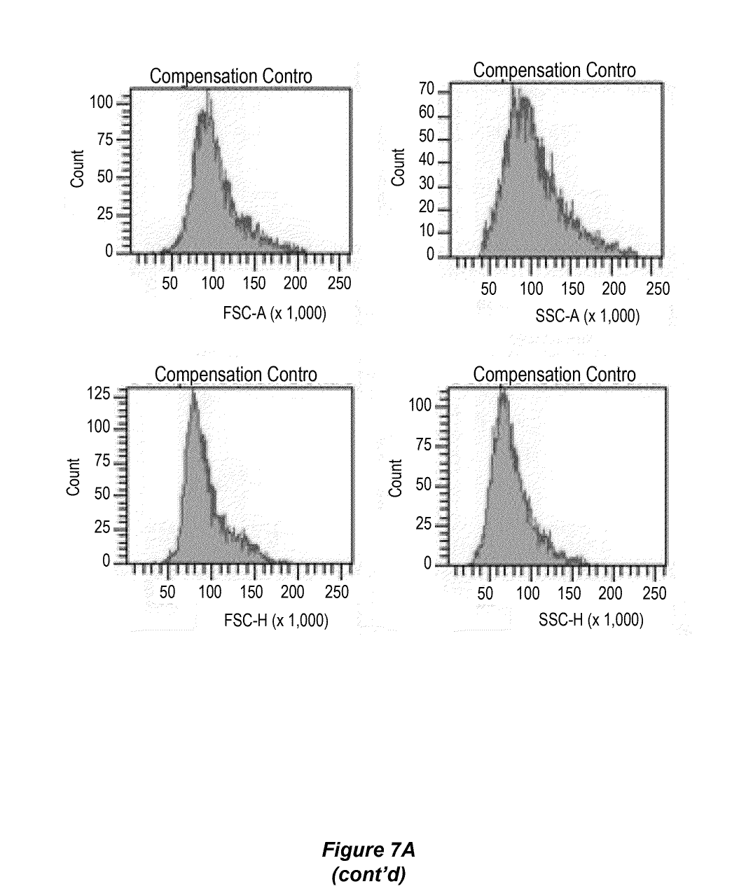

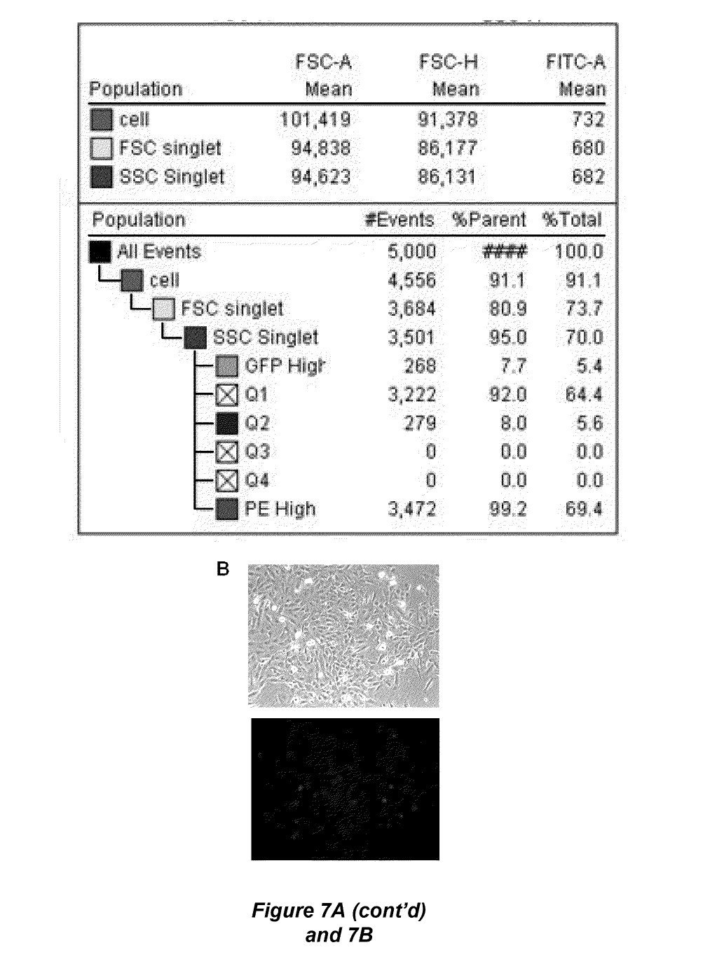

FIG. 7(A) shows a workflow for the sorting of GFP positive and CD9O-PE labeled hfBMSC. The sorting was done to enrich for cells with the desired characteristics. FIG. 7(B) shows a single colony identified from sorted cell population (10× objective)

To see of the MSC had retained the functional property of stem cells, assays were run to determine whether they could be differentiated into mature cell types.

### Multilineage Differentiation

Osteogenic induction: 10000 cells/well are seeded onto 24 well plate, and cultured in completed α-MEM until confluent. Cells are further incubated in the presence of osteogenic medium or completed medium as control for 17 and 21 days respectively. At the day of 17- or 21-day, the cells are washed with PBS and fixed the cells with freshly prepared 4% paraformaldehyde for 15 minutes. Calcium nodule formation is assessed by staining with 0.5% (w/v) Alizarin Red (pH 4.1) for 60 min. The plate is allowed for air dry for 1-2 days and photos are taken under light microscope.

Adipogenic induction: 10000 cells/well are seeded onto 24 well plate, and cultured in completed α-MEM until confluent. Cells are further incubated in the presence of adipogenic medium or completed medium as control for 17 and 21 days respectively. At the day of 17- or 21-day, the cells are washed with PBS and fixed the cells with freshly prepared 4% paraformaldehyde for 15 minutes. Oil droplet formation is assessed by staining with 0.3% (w/v) fresh Oil Red-O solution for 2 h. The plate is allowed for air dry for 1-2 days and photos are taken under light microscope.

Chondrogenic induction: Cell suspension is washed twice with PBS. 1×10⁶ cell pellet is prepared by centrifugation at 450 g for 10 min in 15 ml tube. The pellet with chondrogenic medium or completed medium as control for 21- or 28-day respectively (loosely capped). For the histology study, the pellets are fixed in freshly prepared 4% paraformaldehyde for 1 day, and dehydrated with 70% ethanol, and embedded in paraffin. The embedded cell pellet is sectioned at a 5 μm thickness, and stained with Safranin-O/fast green after deparaffination, and viewed under microscope.

FIG. 8 shows osteogenic and adipogenic differentiation ability of SV40-TK-GFP human fetal BMSC (right column) after 21 days induction in corresponding induction medium, and comparison to its un-transduced counterparts.

Proliferative capacity of the cells was determined using a bromodeoxyuridine (BrdU) incorporation assay. 2000 cells are seeded in 96-well plates and cultured for 24 h, 48 h, and 72 h respectively. 2. 100 μl 1× BrdU in completed medium is added into the cells at different time points and cultured for further 24 h. The medium is removed and 200 μl of Fixation agent is added into each well for 30 min at room temp. The Fixation agent is removed completely and the cells are incubated with 200 μl of Anti-BrdU antibody in working solution for 90 min. The cells are washed with PBS for 3 times. Substrate is added for 20 min incubation at dark. The reaction is stopped by adding 25 μl of 1M H₂SO₄ and the BrdU intensity is measured by a microplate reader at 450 nm and reference at 690 nm.

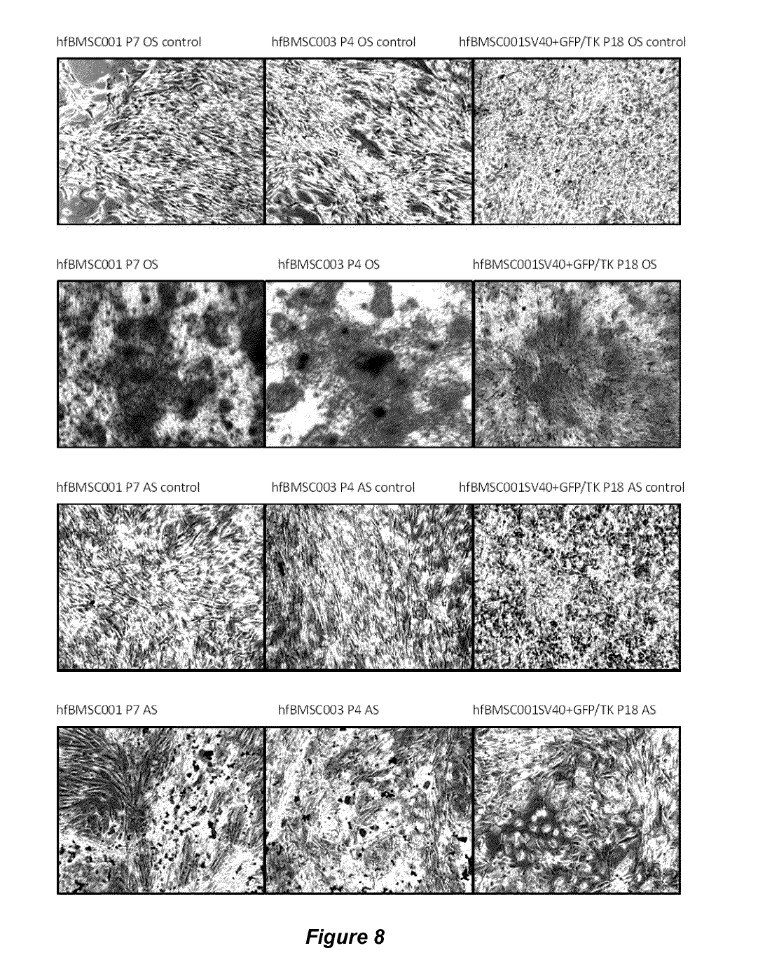

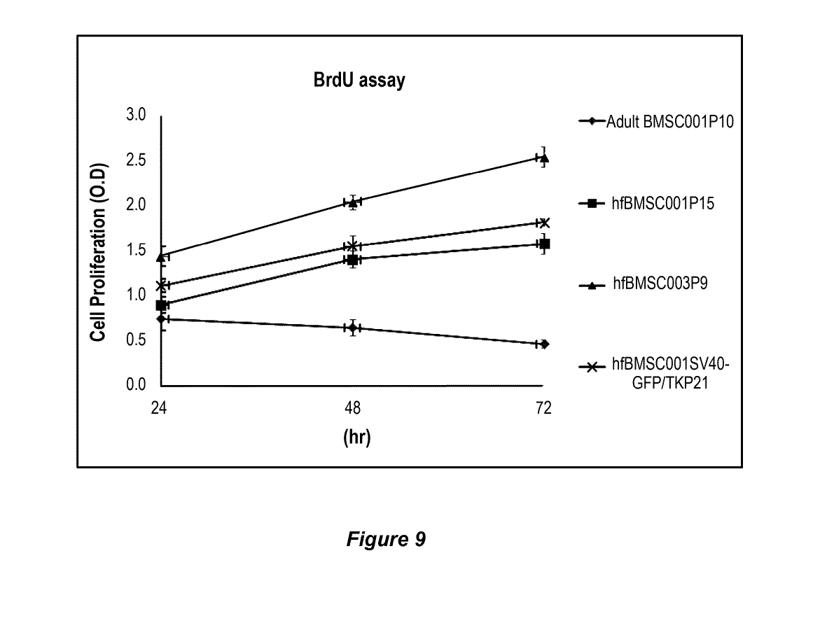

FIG. 9 shows cell proliferation that was observed. Results are mean±SD of three independent experiments. Fetal MSC lines and lines transduced with the GFP/TK vector proliferated substantially over the 3 days of the experiment, whereas an adult MSC line did not.

### Example 4

### Retention of Tumor Homing by Transfected MSC

In vitro cell migration assays were conducted to determine whether the transfected cells retained their natural ability to home to tumor cells.

2×10⁵ cells of DU145 or PC3 cancer cells (these cells have been genetically modified and contained Luciferase and enhanced GFP) are seeded onto bottom chambers with 600 μl completed α-MEM and culture at 37° C. for 24 h. On the next day, 50000 cells (in a 50000 cells/100 μl medium density) of normal MSC or SV40-eGFP/TK transduced MSC are seeded onto transwell inserts with 600 μl completed α-MEM and cultured at 37° C. for 1 h. The completed α-MEM is changed into 1% FBS and 5% PSN α-MEM (600 μl) of the bottom chambers containing DU145 or PC3 cancer cells.

The transwell inserts containing the normal or SV40-GFP/TK transduced MSC is transferred into the bottom chambers and cultured at 37° C. for 16 h. 1% FBS and 5% PSN α-MEM (600 μl) only is used in the bottom chamber of a control group to measure the background migration of hfBMSC. Cells remaining attached to the upper surface of the transwell inserts are carefully removed with cotton swabs. Cells that have migrated to the lower surface of the transwell inserts are fixed with freshly prepared 4% paraformaldehyde. The transwell inserts are washed in PBS and stained with Crystal violet solution for 15 min. The transwell inserts are washed in PBS and allowed for air-dry for 1-2 days. The filters of the transwell inserts are cut and mounted on glass slides. Cells are selected in 5 random fields on each filter and the total number of cells from all fields is calculated.

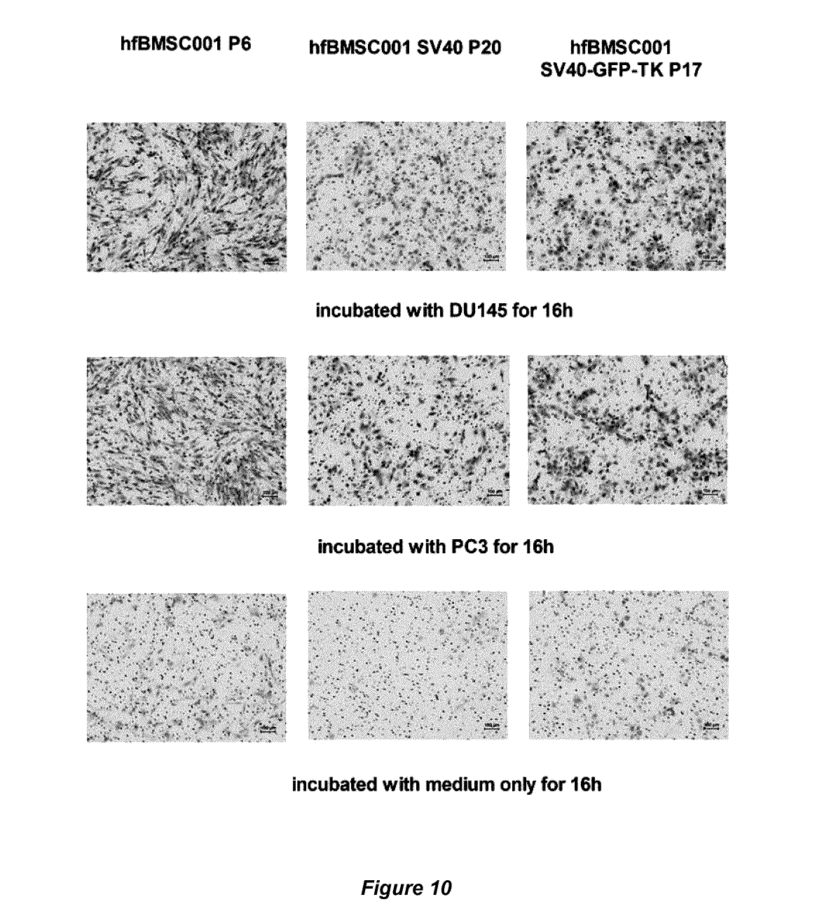

FIG. 10 shows results of a study of the transwell migration ability of hfBMSC in the presence or absence of human prostate cancer cells.

### Example 5

### In Vitro Anti-Tumor Effect

The ability of the transduced MSC to express thymidine kinase from the TK gene, and thereby convert the produg gancyclovir (GCV) to its lethal counterpart can be evaluated by the following assay systems:

GCV toxicity by MTT assay: 5000 cells/well of SV40-TK-GFP transduced MSC, Luc-GFP-DU145, or Luc-GFP-PC3 cells are seeded into 96-well plate respectively and cultured overnight. Every three days, the medium is changed and GCV at different concentrations, ranging from 0, 0.1, 1, 10, 50, to 100 μg/ml, in complete α-MEM is added into the cells and cultured for 3- or 6-days. At day 6, the medium is changed to complete α-MEM supplemented with 0.5 mg/ml MTT. 3 h after incubation, the MTT-containing medium is removed and MTT precipitate is dissolved by addition of 100 μl DMSO. Absorbance is measured by a microplate reader at 570 nm and 630 nm as a reference wavelength.

Anti-cancer effect of MSC on cancer cells in the presence of GCV: 5000 cells of DU145 or PC3 are co-cultured with SV40-TK-GFP transduced MSC at ratio of 1:2, 1:1, 2:1, 5:1, 10:1 or 50:1, or cancer cells alone or with SV40-TK-GFP transduced MSC conditioned medium in 96 well plate. After overnight culture, GCV at different concentrations (0, 1, 10, and 100 μg/ml) are added into the cell culture for 6 days. GCV is replenished every three days. At day 6, the medium is replaced by MTT (0.5 mg/ml) containing medium and the cells are cultured for additional 3 h. The medium is removed and MTT precipitate is dissolved by 100 μl DMSO. Absorbance is measured by a microplate reader at 570 nm and 630 nm as a reference wavelength.

GCV toxicity by Luciferase assay. 5000 cells/well of SV40-TK-GFP transduced MSC, Luc-GFP-DU145, or Luc-GFP-PC3 cells are seeded into 96-well plate respectively and cultured overnight. Every three days, the medium is changed and GCV at different concentrations, ranging from 0, 0.1, 1, 10, 50, to 100 μg/ml, in complete α-MEM is added into the cells and cultured for 3- or 6-days. At day 6, the medium is changed to complete α-MEM supplemented with 150 μg/ml D-Luciferin. 10 min after incubation, the cell viability represented by Luciferase activity is measured using the IVIS 200 systems.

Anti-cancer effect in the presence of GCV by Luciferase assay: 5000 cells of DU145 or PC3 are co-cultured with transduced MSC at ratio of 1:5, 1:1, 5:1, or cancer cells alone or with SV40-TK-GFP transduced MSC conditioned medium in 96 well plate. After overnight culture, GCV at different concentrations (0, 10, 50, and 100 μg/ml) are added into the cell culture for 6 days. GCV is replenished every three days. At day 6, the medium is changed to complete α-MEM supplemented with 150 μg/ml D-Luciferin. 10 min after incubation, the cell viability represented by Luciferase activity is measured using the IVIS 200 systems.

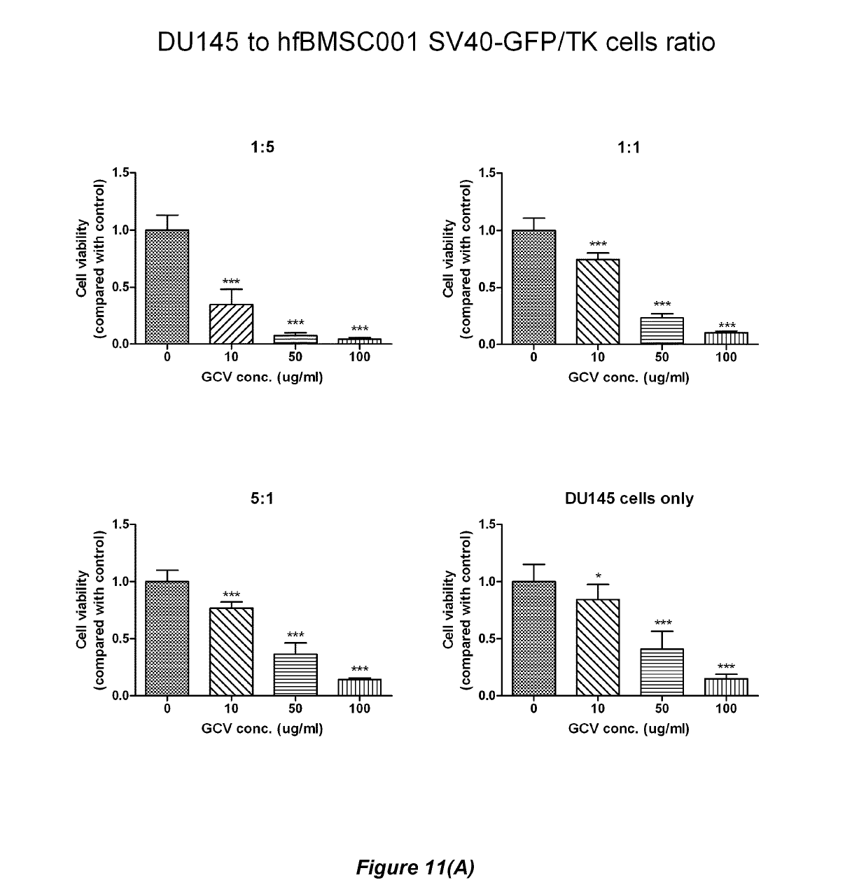

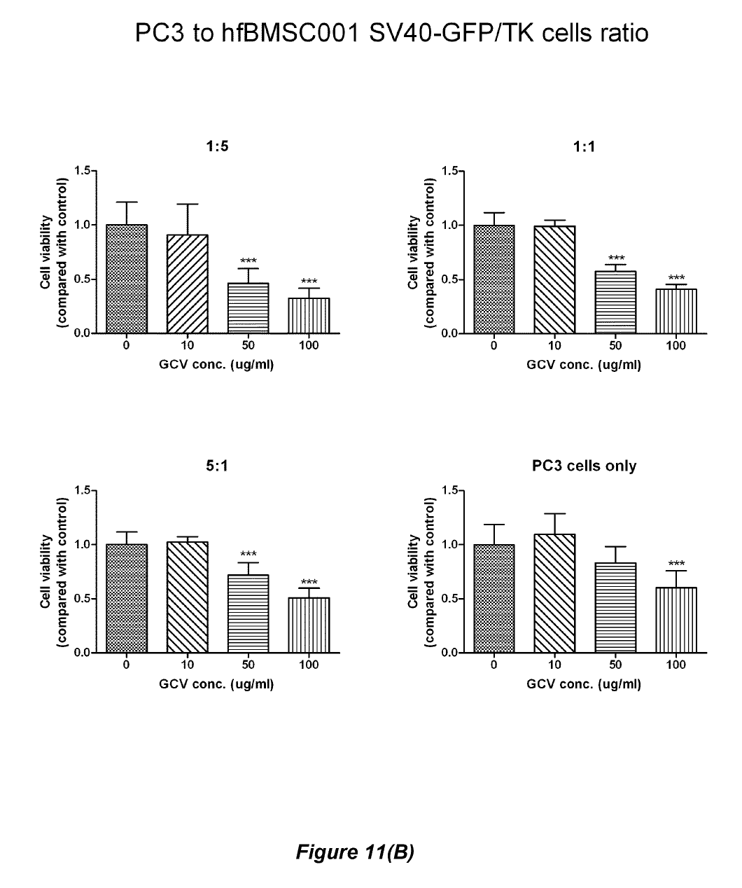

FIG. 11(A) shows in vitro cytotoxicity of GCV on human prostate cancer cell line (DU145) in the co-culture of SV40-TK-GFP human fetal BMSC. Cell viability was measured and indicated by the activity of luciferase expressed in the cancer cell lines via IVIS 200. Data is mean±SD of three independent experiments. Statistics was performed by one way ANOVA with Tukey's Post hoc test. * p<0.05; *** p<0.001. FIG. 11(B) shows in vitro cytotoxicity of GCV on human prostate cancer cell line (PC3) in the co-culture of SV40-TK-GFP human fetal BMSC. Cell viability was measured and indicated by the activity of luciferase expressed in the cancer cell lines via IVIS 200. Data is mean±SD of three independent experiments. Statistics was performed by one way ANOVA with Tukey's Post hoc test. *** p<0.001.

### Example 6

### Immunogenic Features

Mixed human peripheral blood lymphocyte culture (an MLR assay) was run to determine immunoreactivity of transduced MLR cells.

Day 1: In 96-well plates, 5000, 10000, and 20000 undifferentiated MSC (hfBMSC001 or 001SV40+GFP/TK, stimulators) are seeded into each well for 24 h. Day 2: Add osteogenic, adipogenic or chondrogenic stimulation medium into the cells respectively and normal medium as a control for 7 days. All the stimulator cells are treated with 2.5 μg/ml mitomycin C during induction culture. Day 8: At the end of differentiation treatment, cells are washed twice with PBS, and 1×10⁵ hPBLs (responders) isolated from healthy donors are then added into each well. Culture MSC alone without adding the hPBLs as a negative control, hPBLs alone as a baseline control, and hPBLs are treated with 100 ng/ml LPS for 18 h as a positive control. Day 13: The proliferation rate of hPBLs is assessed by Bromodeoxyuridine (BrdU) incorporation assay (Roche BrdU kit), manual according to the manufacturer. Day 14: Absorbance is measured at 450 nm and at 690 nm (reference) using a microplate reader.

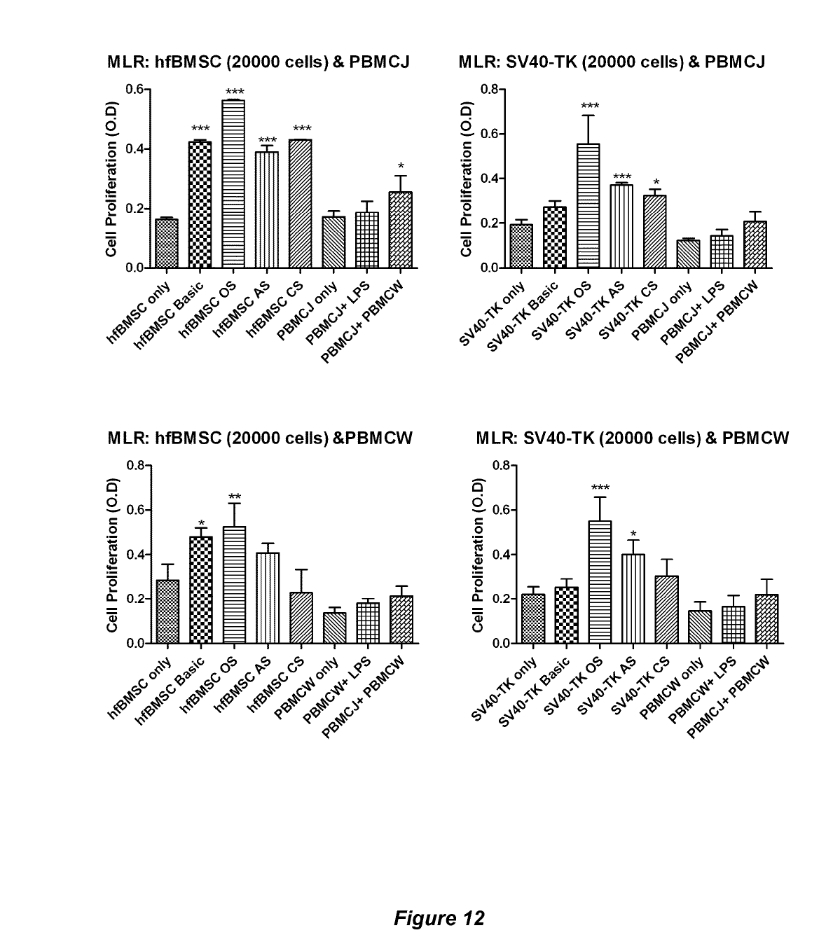

FIG. 12 shows results of an MLR assay on the transduced MSC. The lymphocytes were collected from two consented healthy male Asians denoted as PBMCJ and PBMCW. Data is mean±SD of three independent experiments. Statistics was performed by one way ANOVA with Tukey's Post hoc test. * p<0.05; ** p<0.01; *** p<0.001.

### Example 7

### In Vivo Anti-Tumor Effect

In vivo implantation can be done as a biosafety test. 1.5×10⁶ MSC or gene transduced MSCs are mixed with 40 mg HA/TCP powder for 2 h to allow cell attachment. MSCs-HA/TCP powder is then implanted subcutaneously into the dorsal side of 12-week-old male nude mice. At 8 weeks post implantation, the implants are harvested and fixed with 10% neutral buffered formalin, decalcified with buffered 9% formic acid for 2-3 weeks, and embedded in paraffin for histological examination. The sign of tumor formation and other tissue formation (such as bone and cartilage) is examined.

In vivo anti-tumor effect was determined as follows: 5×10⁶ cells of Luc-GFP-DU145 or Luc-GFP-PC3 are pelleted and inoculated at the dorsal site of nude mice (22 g) subcutaneously. The viability of the cell pellet is determined by Luciferase assay. After 4 weeks, 1×10⁶ cells of SV40-TK-GFP transduced MSC is injected intratumorally. After 2 recovery days, 60 mg/kg GCV is injected subcutaneously at the dorsal site for 5 consecutive days. Steps 2 and 3 are repeated once more. The change in tumor size along the treatment is measured by caliper, and calculated as length×width²/2.

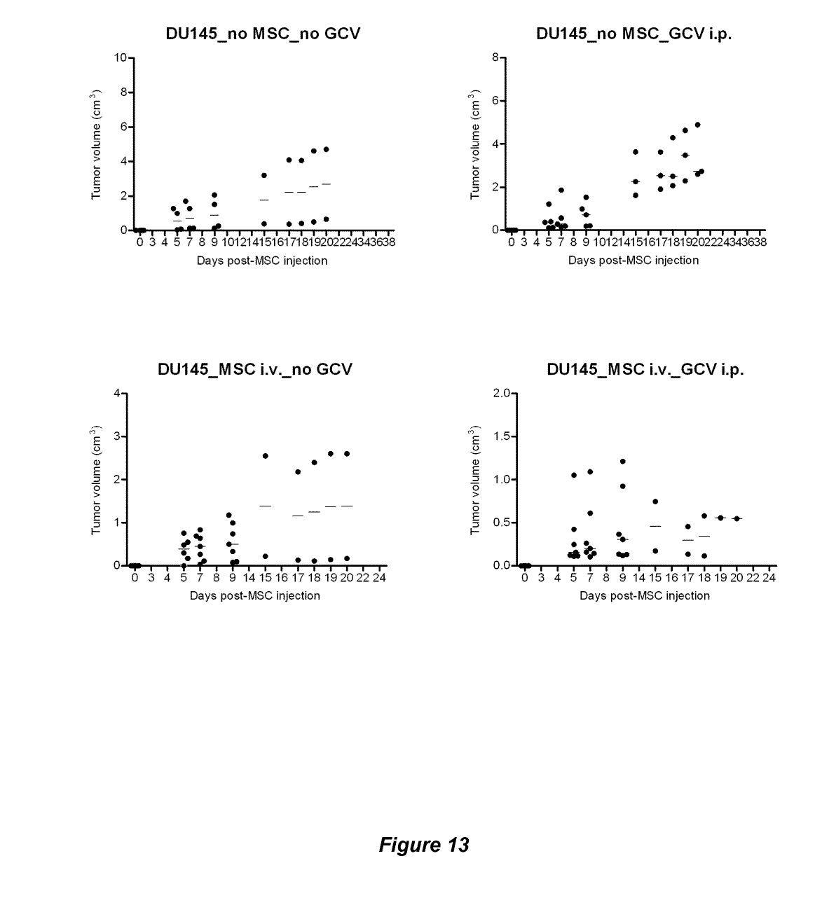

FIG. 13 shows in vivo anti-tumor effect in an experiment where there was a systemic injection of transduced BMSC and an intraperitoneal injection of GCV. The subjects were nude mice bearing human prostate cancer cells (DU145).

For all purposes in the United States of America, each and every publication and patent document cited herein is incorporated herein by reference as if each such publication or document was specifically and individually indicated to be incorporated herein by reference.

The examples that follow are provided by way of further illustration, and are not meant to imply any limitation in the practice of the claimed invention or its equivalents.

1. A method for treating a tumor in a subject, comprising:
a) administering to the subject a population of immortalized human fetal mesenchymal stem cells that recombinantly express a suicide gene;
b) permitting at least some of the mesenchymal stem cells to migrate to a location at or around the tumor; and then
c) administering to the subject an effective amount of a prodrug, whereby expression of the suicide gene by the mesenchymal stem cells that are located at or around the tumor causes the prodrug to be converted to a drug that is lethal to cells of the tumor.

2. The method of claim 1, wherein the mesenchymal stem cell population is pleiotropic.

3. (canceled)

4. The method of claim 1, wherein the mesenchymal stem cells have been immortalized by transfecting with SV40 large T antigen.

5. (canceled)

6. The method of claim 1, wherein the suicide gene is a thymidine kinase.

7. The method of claim 6, wherein the prodrug is ganciclovir.

8. A line of pleiotropic immortalized human fetal mesenchymal stem cells that recombinantly express a suicide gene.

9. (canceled)

10. The cell line of claim 8, wherein the mesenchymal stem cells have been immortalized by transfecting with lentivirus.

11. (canceled)

12. The cell line of claim 8, wherein the suicide gene is a thymidine kinase.

13. A pharmaceutical composition for treating cancer, comprising a line of mesenchymal stem cells according to claim 8, and a physiologically compatible buffer.

14. A kit for treating a tumor in a subject, comprising:
a) a pharmaceutical composition comprising the line of pleiotropic immortalized human fetal mesenchymal stem cells of claim 8, and
b) a prodrug, wherein expression of the suicide gene in the mesenchymal stem cells of the composition causes the prodrug to be converted to a drug that is lethal to tumor cells.

15. The kit of claim 14, wherein the suicide gene is thymidine kinase, and the prodrug is ganciclovir.
